(19) United States
(12) Patent Application Publication
(10) Pub. No.: US 2006/0095100 A1
(43) Pub. Date: May 4, 2006

(54) Method and apparatus for regulating light administered at a patient treatment site

(76) Inventors: Kian Shin Lee, Penang (MY); Thye Linn Mok, Penang (MY); Kheng Leng Tan, Penang (MY); Fakhrul Arifin Mohd Afif, Prai Penang (MY); Siew It Pang, Penang (MY); Yew Cheong Kuan, Penang (MY); Norfidathul Aizar Abdul Karim, Seberang Perai (MY); Su Lin Oon, Penang (MY); Wen Ya Ou, Penang (MY); Sundar A/L Natarajan Yoganandan, Pulau Penang (MY)

Correspondence Address: AGILENT TECHNOLOGIES, INC.;INTELLECTUAL PROPERTY ADMINISTRATION, LEGAL DEPT., P.O. BOX 7599, M/S DL429, LOVELAND, CO, 80537-0599, US

(21) Appl. No.: 10/977,925

(22) Filed: Oct. 29, 2004

(51) Int. Cl. A61N 5/06

(52) U.S. Cl. 607/89

(57) ABSTRACT

In one embodiment, apparatus is provided with at least one solid-state light emitter, at least one fiber optic light guide, at least one photosensor, and a control system. Each of the at least one fiber optic light guide has a first end that is independently positionable with respect to the solid-state light emitter(s) to receive the light emitted by the solid-state light emitter(s). Each photosensor is positioned to receive light emitted from a second end of one of the light guides. The control system is operably associated with the solid-state light emitter(s) and the photosensor(s) to regulate the light output of the light emitter(s) in accordance with measurements received from the photosensor(s).

### BACKGROUND

Photodynamic therapy (PDT) is a clinical treatment in which a photoreactive agent is typically injected or applied at a patient treatment site (e.g., a tumor), and then activated by light emitted from a light source. In some cases, the light source may be a light bar on which a plurality of solid-state light emitters (e.g., light emitting diodes (LEDs)) are mounted. When activated by the light, the photoreactive agent may help to cure a problem at the treatment site. For example, the photoreactive agent may target and destroy unhealthy cells such as cancer cells. If the agent is primarily isolated at the treatment site, or if the light is primarily directed toward the treatment site, damage to healthy tissue surrounding the treatment site may be limited.

In some cases, photodynamic therapy may be administered without a photoreactive agent. That is, certain wavelengths of light, administered alone, may also help to cure a problem at the treatment site.

One exemplary light source that may be used in photodynamic therapies is the LitX™ platform offered by Light Sciences Corporation (having its principal place of business in Snoqualmie, Wash., USA).

### SUMMARY OF THE INVENTION

In one embodiment, apparatus for regulating light administered at a patient treatment site comprises at least one solid-state light emitter, at least one fiber optic light guide, at least one photosensor and a control system. The light emitter(s) are provided to be positioned adjacent the treatment site. The light guide(s) each have a first end to receive light emitted by the solid-state light emitter(s). The photosensor(s) are each positioned to receive light emitted from a second end of one of the light guides. The control system is operably associated with the solid-state light emitter(s) and the photosensor(s), to regulate the light output of the light emitter(s) in accordance with measurements received from the photosensor(s).

In another embodiment, apparatus comprises at least one solid-state light emitter, at least one fiber optic light guide, at least one photosensor and a control system. The light guide(s) each have a first end that is independently positionable with respect to the solid-state light emitter(s), to receive light that is emitted by the solid-state light emitter(s). The photosensor(s) are each positioned to receive light emitted from a second end of one of the light guides. The control system is operably associated with the solid-state light emitter(s) and the photosensor(s), to regulate the light output of the light emitter(s) in accordance with measurements received from the photosensor(s).

In yet another embodiment, a method comprises positioning at least one solid-state light emitter adjacent a patient treatment site. A first end of at least one fiber optic light guide is then positioned to receive light emitted by the solid-state light emitter(s). Via a control system, the solid-state light emitter(s) are activated; at least one photosensor is activated to measure light emitted from a second end of one of the fiber optic light guides; and the light emitted from the solid-state light emitter(s) is regulated in accordance with the measurements taken by the photosensor(s).

In an additional embodiment, a method for activating a photoreactive agent at a patient treatment site comprises applying a photoreactive agent to the treatment site (with the photoreactive agent being activated by one or more wavelengths of light). At least one solid-state light emitter, capable of emitting the one or more wavelengths of light, is then positioned adjacent the treatment site; and a first end of at least one fiber optic light guide is positioned to receive light emitted the solid-state light emitter(s). The control system is then activated to, in turn, 1) activate the solid-state light emitter(s), 2) activate at least one photosensor to measure light emitted from a second end of one of the fiber optic light guides, and 3) tune the light emitted from the solid-state light emitter(s) to the one or more wavelengths, in accordance with the measurements taken by the photosensor(s).

Other embodiments are also disclosed.

### BRIEF DESCRIPTION OF THE DRAWINGS

Illustrative and presently preferred embodiments of the invention are illustrated in the drawings, in which:

FIG. 1 illustrates an exemplary method for regulating light administered at a patient treatment site;

FIG. 2 illustrates exemplary apparatus for regulating light administered at a patient treatment site;

FIG. 3 illustrates placement of the light emitter(s) and light guide(s) of the FIG. 2 apparatus in the vicinity of a patient treatment site, such as a tumor;

FIG. 4 illustrates an exemplary light bar carrying the solid-state light emitters of the apparatus shown in FIG. 2; and

FIG. 5 illustrates a method for activating a photoreactive agent at a patient treatment site.

### DETAILED DESCRIPTION OF AN EMBODIMENT

Solid-state light emitters (e.g., LEDs) are particularly useful in PDT systems because they are inexpensive to manufacture, do not contain glass or other highly fragile materials, are widely available, and do not generate a lot of heat. However, the physical and electrical characteristics of solid-state light emitters (e.g., turn-on voltage) can vary from batch to batch, leading to nominally identical LEDs having different optical properties. Furthermore, the optical properties of LEDs can change or deteriorate with factors such as changes in temperature and age. As a result, in PDT systems where the integrity of light emitted by a light source needs to be maintained, it would be useful to have some sort of means for regulating the light source's light.

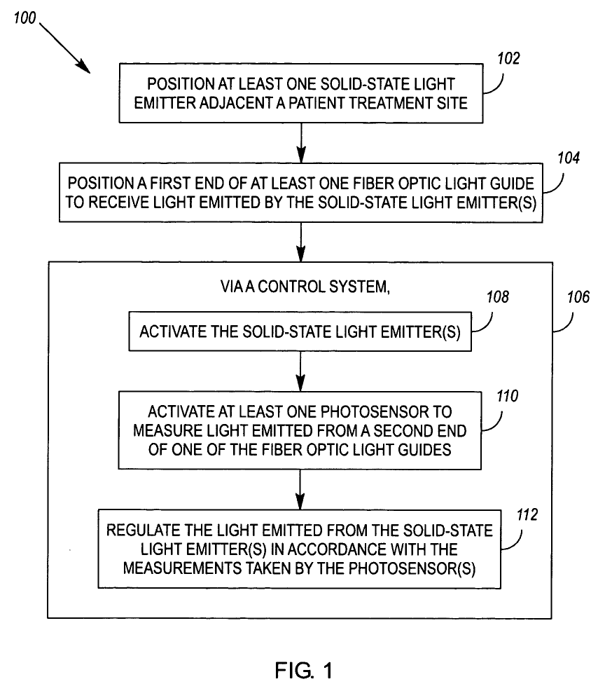

FIG. 1 illustrates an exemplary method 100 for regulating light (A, FIG. 3) administered at a patient treatment site 300. In accordance with the method 100, at least one solid-state light emitter 202, 204 is positioned 102 adjacent the treatment site 300. By way of example, the solid-state light emitters 202, 204 may comprise a plurality of LEDs that are mounted on a substrate 206 to form a light bar 208.

The method 100 continues with the positioning 104 of a first end 210 of at least one fiber optic light guide 212 to receive light emitted by the solid-state light emitter(s) 202, 204. The first end(s) 210 of the light guide(s) 212 may be positioned adjacent the light emitter(s) 202, 204, the treatment site 300 or both.

After positioning 102, 104 the light emitter(s) 202, 204 and light guide(s) 212, a control system 214 may be used 106 to activate 108 the light emitter(s) 202, 204. The control system 214 may also 1) activate 110 at least one photosensor 216, 218, 220 to measure light emitted from a second 222 end of one of the light guides 212, and 2) regulate 112 the light emitted from the light emitter(s) 202, 204 in accordance with the measurements taken by the photosensor(s) 216-220. By way of example, the photosensor(s) 216-220 may comprise one or more photodiodes or phototransistors that measure the intensity of one or more wavelengths of light.

In one embodiment, the control system 214 may regulate the intensity of light emitted from the light emitter(s) 202, 204, in accordance with a light intensity measurement obtained from a single photosensor. For example, the control system 214 may compare a light intensity obtained from the photosensor with a desired intensity, and then adjust the drive signal(s) of the light emitter(s) if the measured intensity is out of range. This method of regulation is especially useful if the light emitter(s) all emit the same wavelength of light (e.g., if all of the light emitters are red LEDs).

In another embodiment, the control system 214 may regulate the color of light emitted from the light emitter(s) 202, 204, in accordance with light intensity measurements obtained from plural photosensors 216-220 (e.g., red, green and blue photosensors), or in accordance with a single photosensor that is serially filtered to detect different wavelengths of light (e.g., a photosensor behind a color wheel). For example, the control system 214 may compare each of the light intensity measurements obtained from the photosensors 216-220 with a plurality of intensities corresponding to a desired color and, if a measured intensity is out of range, the control system 214 may adjust the drive signal(s) of a corresponding color of light emitter 202. This latter method of regulation is especially useful if the different light emitters 202, 204 emit two or more different wavelengths of light.

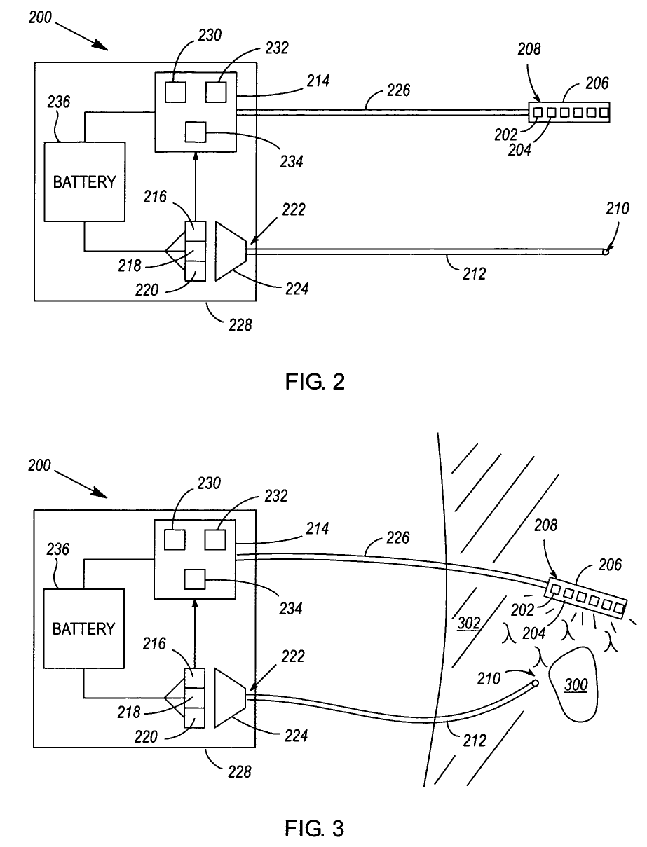

As partially introduced above, FIG. 2 illustrates exemplary apparatus 200 for regulating light administered at a patient treatment site 300 (see FIG. 3). The apparatus 200 comprises at least one solid-state light emitter 202, 204 to be positioned adjacent the treatment site 300. If the apparatus 200 comprises a plurality of light emitters 202, 204, different light emitters 202, 204 may emit the same or different wavelengths of light. If the apparatus 200 comprises only one light emitter, or a plurality of light emitters of the same color, then only the intensity of the light source 208 may be regulated (as previously described). If the light emitters 202, 204 are of different colors, then both the intensity and color of the light source 208 may be regulated.

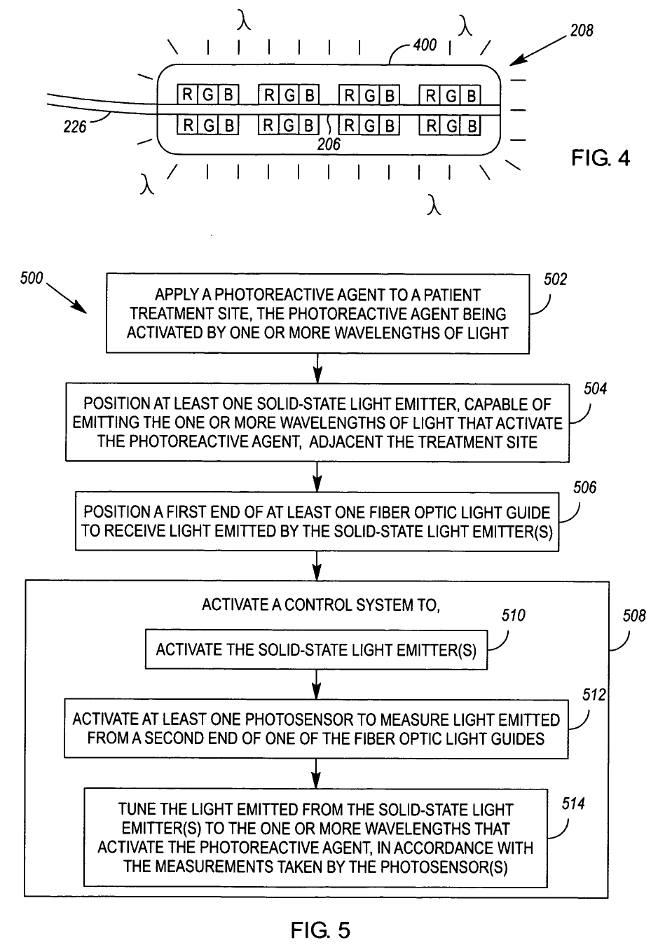

The light emitters 202, 204 may variously comprise LEDs, laser diodes, or other solid-state devices. As shown in FIG. 4, the light emitters may comprise red (R), green (G) and blue (B) light emitters arranged in RGB triads on both sides of a substrate 206, thereby forming a light bar 208. The light emitters could alternately be arranged in other patterns, or only one side of the substrate.

The light bar 208 may be surrounded by a lens, light mixer sheath 400 or other element to encapsulate the light emitters and 1) provide a protective barrier between the circuitry of the light bar 208 and a patient 302, and 2) mix the light emitted by different-colored light emitters. In some cases, a light mixer sheath may comprise a diffusant or coating (e.g., a titanium oxide or silicon oxide coating) that serves to diffuse light. In other cases, a paper, gel or encapsulant-type diffuser could be provided between the light bar 208 and the sheath 400.

The apparatus 200 also comprises at least one fiber optic light guide 212, at least one photosensor 216-220, and a control system 214. For each fiber optic light guide 212, a first end 210 of the light guide is positioned to receive light (A) emitted by the light emitter(s) 202, 204. In addition to receiving the light emitted by the light emitter(s) 202, 204, the fiber optic light guide(s) 212 may serve to mix and/or filter light. If a plurality of light guides are provided, they could be enclosed in a common sheath.

Each photosensor 216-220 may be positioned to receive light emitted from a second end 222 of one of the light guides 212. In one embodiment, the apparatus 200 comprises only a single fiber optic light guide 212, with one or more photosensors 216-220 being positioned to receive light from the single fiber optic light guide 212. To disperse the light emitted from the light guide 212 to multiple photosensors 216-220, a diffuser 224 or one or more lenses may be positioned between the light guide 212 and the photosensors 216-220. Alternately, only a single photosensor could be used to sense the light emitted from the light guide. In this latter case, the single photosensor could sense different wavelengths of light by replacing the diffuser 224 with a color wheel (or some other element to differently and serially filter the light emitted from the light guide). Alternately, different photosensors may be positioned to receive light from different light guides. Mounting the photosensor(s) 216-220 to receive light from the end(s) 222 of light guide(s) 212 opposite the end(s) 210 positioned near a treatment site 300 can be advantageous in that the photosensor(s) 216-220 and their associated electronics do not block the light (λ) being administered at the treatment site 300.

In one embodiment, the photosensors 216-220 comprise two or more photosensors, each having a differently filtered input to measure a different wavelength (or range of wavelengths) of light. For example, different photosensors could respectively measure red, green and blue wavelengths of light.

The fiber optic light guide(s) 212 may be naturally flexible. Likewise, the light emitter(s) 202, 204 may be coupled to an elongate flexible member 226. The light emitter(s) 202, 204 and light guide(s) 212 are preferably independently positionable with respect to one another. In this manner, a physician is provided with more versatility in positioning the light emitter(s) 202, 204 and light guide(s) 212, possibly allowing the physician to position the light guide(s) 212 closer to the treatment site 300 than is possible to maneuver a light bar 208 or the like. This, in turn, enables the light guide(s) 212 to sense light as it is actually being administered to the treatment site 300. Alternately, the light emitter(s) 202, 204 and light guide(s) 212 may be fixed with respect to one another (e.g., via a bracket, or by attaching the light guide(s) 212 to the light bar 208). In one embodiment, the light guide(s) 212 and wiring for the light bar 208 may be enclosed within a common sheath, and the light guide(s) may be attached to the substrate 206 of the light bar 208.

As shown in FIG. 2, the photosensor(s) 216-220 and control system 214 may be mounted within a common housing 228. The light guide(s) 212 may then be coupled to the housing 228 such that the photosensor(s) 216-220 can receive light carried by the light guide(s) 212. The elongate flexible member 226 coupled to the light emitter(s) 202, 204 may also be coupled to the housing 228, and the electrical connections carried therein may then be coupled to the control system 214. Likewise, the photosensor(s) 216-220 may be coupled to the control system 214. In this manner, the control system 214 may receive measurements from the photosensor(s) 216-220 and, in turn, regulate the light output of the light emitter(s) 202, 204 (e.g., by regulating their drive signals).

In one exemplary embodiment, the control system 214 may comprise driver circuitry 230, a color management system 232, and a microcontroller 234. The physical boundaries between these functional components 230-234 are somewhat arbitrary, and the functionality of the components 230-234 could be merged or divided into a fewer or greater number of components. In use, the color management system 232 receives desired color and/or intensity settings from the microcontroller 234, and converts the color setting (if provided) to a plurality of intensity settings for different colored light emitters. The color management system 232 also receives intensity measurements from the photosensor(s) 216-220. The color management system 232 then compares corresponding intensity measurements and, if a measurement is out of range, it adjusts the light output of a corresponding light emitter 202 by, for example, modulating its drive current via the driver circuitry 230.

By raising or lowering the drive currents of all light emitters 202, 204 in unison, the color management system 232 can control the intensity of a light source 208. By adjusting the ratios of drive currents supplied to different-colored light emitters 202, 204, the color management system 232 can control the color of the light source 208.

The apparatus 200 shown in FIG. 2 may comprise various additional components, including a power supply 236 (e.g., a battery) to supply power to the control system 214, photosensor(s) 216-220 and light emitter(s) 202, 204. Alternately, the apparatus 200 may be powered by a remote power source (e.g., alternating current from a wall jack).

FIG. 3 illustrates placement of the light emitter(s) 202, 204 and light guide(s) 212 in the vicinity of a patient treatment site 300, such as a tumor. Once placed, the control system 214 may be activated or programmed to control the light that is administered to the treatment site 300. In some cases, the control system 214 may simply maintain a constant light of a given color. In other cases, the control system 214 may cycle the administered light through a sequence of ON and OFF cycles. In still other cases, the control system 214 may cycle the administered light through a series of different colors (perhaps, as different photoreactive agents are injected into the treatment site 300).

As shown in FIG. 3, the housing 228 for the control system 214, photosensor(s) 216-220 and power supply 236 may be located externally to a patient 302, and then operated by a physician or the patient. Alternately, the housing 228 might be implanted within the patient, and programmed before or after implantation. By way of example, programming after implementation could be accomplished via an electromagnetic programming device.

FIG. 5 illustrates a method 500 for activating a photoreactive agent at a patient treatment site. The method 500 comprises applying 502 a photoreactive agent to the treatment site (the agent being activated by one or more wavelengths of light). At least one solid-state light emitter, capable of emitting the one or more wavelengths of light that activate the photoreactive agent, is then positioned 504 adjacent the treatment site. The first end of at least one fiber optic light guide is then positioned 506 to receive light emitted by the solid-state light emitter(s). Thereafter, a control system is activated 508 to 1) activate the light emitter(s), 2) activate at least one photosensor to measure light emitted from a second end of the at least one fiber optic light guide, and 3) tune the light emitted from the solid-state light emitter(s) to the one or more wavelengths that activate the photoreactive agent. In some cases, the control system may tune the light by adjusting its intensity. In other cases, the control system may tune the light by adjusting its color.

What is claimed is:

1. Apparatus for regulating light administered at a patient treatment site, comprising:
at least one solid-state light emitter to be positioned adjacent the treatment site;
at least one fiber optic light guide, each having a first end to receive light emitted by the solid-state light emitter(s);
at least one photosensor, each positioned to receive light emitted from a second end of one of the light guides; and
a control system, operably associated with the solid-state light emitter(s) and the photosensor(s), to regulate the light output of the light emitter(s) in accordance with measurements received from the photosensor(s).

2. The apparatus of claim 1, wherein the solid-state light emitter(s), photosensor(s) and control system are battery-operated.

3. The apparatus of claim 1, wherein the at least one solid-state light emitter comprises a plurality of solid-state light emitters that emit two or more different wavelengths of light.

4. The apparatus of claim 1, wherein the solid-state light emitter(s) and fiber optic light guide(s) are independently positionable with respect to each other.

5. The apparatus of claim 1, wherein the solid-state light emitter(s) and fiber optic light guide(s) are attached to a common substrate.

6. The apparatus of claim 1, wherein the solid-state light emitter(s) are coupled to the control system via an elongate flexible member.

7. The apparatus of claim 1, wherein at least two different photosensors have differently filtered inputs and measure different wavelengths of light.

8. The apparatus of claim 1, wherein the at least one solid-state light emitter comprises red, green and blue light emitters; wherein different photosensors measure red, green and blue wavelengths of light; and wherein the control system separately regulates the intensities of the red, green and blue light emitters in accordance with the measurements received from the photosensors.

9. The apparatus of claim 8, wherein the light emitters comprise light emitting diodes (LEDs).

10. The apparatus of claim 1, wherein the solid-state light emitter(s) comprise one or more light emitting diodes (LEDs).

11. The apparatus of claim 1, wherein the at least one fiber optic light guide is a single fiber optic light guide.

12. The apparatus of claim 11, wherein the at least one photosensor comprises a plurality of photosensors, each measuring a different wavelength of light, and each positioned to receive light from the single fiber optic light guide.

13. Apparatus for regulating light administered at a patient treatment site, comprising:
at least one solid-state light emitter;
at least one fiber optic light guide, each having a first end that is independently positionable with respect to the solid-state light emitter(s) to receive light emitted by the solid-state light emitter(s);
at least one photosensor, each positioned to receive light emitted from a second end of one of the light guides; and
a control system, operably associated with the solid-state light emitter(s) and the photosensor(s), to regulate the light output of the light emitter(s) in accordance with measurements received from the photosensor(s).

14. The apparatus of claim 13, wherein the at least one solid-state light emitter comprises a plurality of solid-state light emitters that emit two or more different wavelengths of light.

15. A method, comprising:
positioning at least one solid-state light emitter adjacent a patient treatment site;
positioning a first end of at least one fiber optic light guide to receive light emitted by the solid-state light emitter(s);
via a control system,
activating the solid-state light emitter(s);
activating at least one photosensor to measure light emitted from a second end of one of the fiber optic light guides; and
regulating the light emitted from the solid-state light emitter(s) in accordance with the measurements taken by the photosensor(s).

16. The method of claim 15, wherein the control system regulates the intensity of light emitted from the solid-state light emitter(s), in accordance with a light intensity measurement obtained from a single photosensor.

17. The method of claim 15, wherein the control system regulates the color of light emitted from the solid-state light emitter(s), in accordance with light intensity measurements obtained from plural photosensors.

18. The method of claim 15, further comprising, positioning the first end of the at least one fiber optic light guide adjacent the solid-state light emitter(s).

19. The method of claim 15, further comprising, positioning the first end of the at least one fiber optic light guide adjacent the treatment site.

20. A method for activating a photoreactive agent at a patient treatment site, comprising:
applying a photoreactive agent to the treatment site, the photoreactive agent being activated by one or more wavelengths of light;
positioning at least one solid-state light emitter, capable of emitting said one or more wavelengths of light, adjacent the treatment site;
positioning a first end of at least one fiber optic light guide to receive light emitted the solid-state light emitter(s); and
activating a control system to,
activate the solid-state light emitter(s);
activate at least one photosensor to measure light emitted from a second end of one of the fiber optic light guides; and
tune the light emitted from the solid-state light emitter(s) to said one or more wavelengths, in accordance with the measurements taken by the photosensor(s).
